(19) United States
(12) Patent Application Publication
(10) Pub. No.: US 2006/0264792 A1
(43) Pub. Date: Nov. 23, 2006

(54) Hand orthosis

(76) Inventor: Karen L. Bonn, Brandenburg, KY (US)

Correspondence Address: JAMES C. EAVES JR.;GREENEBAUM DOLL & MCDONALD PLLC, 3500 NATIONAL CITY TOWER, 101 SOUTH FIFTH STREET, LOUISVILLE, KY, 40202, US

(21) Appl. No.: 11/438,558

(22) Filed: May 22, 2006

Related U.S. Application Data
(60) Provisional application No. 60/683,565, filed on May 23, 2005.

(51) Int. Cl. A61F 5/00

(52) U.S. Cl. 602/21

(57) ABSTRACT

The present invention relates to an othosis or splint which can contain multiple features. All splints contain a moldable thermoplastic insert surrounded by padding material. The insert has slits between the digits of the hand so that the insert can be molded for the individual digits. This padded splint goes underneath the hand, wrist, and forearm. A separate moldable thermoplastic insert, again surrounded by padding can form a dorsal portion to go opposite the splint on the back of the hand, wrist, and forearm creating a “bi-valve” type of splint to address wrist hyperextension, extensor tone or other needs to control this area of the extremity. A plurality of straps attach to the padding of the underneath splint. Cylindrical shaped rolls can be attached to the insert of the splint just proximal to a patient's hyperextended joint(s). These rolls work with elastic straps on the splint to assist with correcting hyperextension of the MCP joints, PIP joints (Proximal Interphalangeal) or DIP joints (Distal Interphalangeal) of the thumb or fingers.

This application claims priority to an incorporates by reference herein U.S. Provisional Patent Application serial No. 60/683,565, filed May 23, 2005.

### BACKGROUND OF THE INVENTION

(a) Field of the Invention

The present invention relates to a hand orthosis or splint used with restorative or rehabilitation patients. An orthosis is an external orthopedic device that prevents or assists the movement of limbs. A restorative orthosis addresses lost range of motion with or without abnormal neurological tone.

(b) Description of the Prior Art

U.S. Pat. Nos. 5,891,068; 6,001,074; 6,064,912; 6,206,846; 6,547,752; and 6,456,884; all listing John P. Kenney as the inventor, teach various orthotic devices.

### SUMMARY OF THE INVENTION

The present invention relates to a hand orthosis or splint used with restorative or rehabilitation patients. These devices are appropriate for either a patient who has need for support of wrist/hand/fingers after surgery or injury, or for a patient who has lost range of motion resulting in shortened adaptive tissue with or without neurological tone, and a patient should have these fit as soon as they begin to develop neurological tone to allow them to work through their tone. The “old” traditional style of hand orthosis are either: 1) designed to remain rigid, which tend to exacerbate any existing tone and therefore causing pain and actually increasing deformities; or 2) styles like wire foam or aluminum foam (a water resistant foam material over either a wire or aluminum frame) that do not allow true therapeutic value. When these designs of hand splints are put on a patient that exhibits tone, the involuntary force exerted by the patient on the wire or aluminum frame causes it to bend to the shape in which the patient pulls it. When fit to a patient who has adaptive tissue shortening, the device offers no required prolonged low load passive stretch to improve the range of motion, but remains in the shape held by the patient's curvature.

Restorative Orthosis have been and still are today a low priority for healthcare since they are perceived by many to be only for the geriatric population. This is not so. As an example, children with Cerebral Palsy or other neurological conditions should be appropriately splinted as soon as they begin to develop neurological tone, and any age person can sustain spinal injury or closed head injury that will result in neurological tone. Not appropriately treating tone of any aged person can result in severe deformities, pain and suffering, and loss of self-esteem.

A rehabilitation patient is one who requires a fast, insurance driven treatment plan and has typically suffered injury or had surgery. A restorative patient is one who requires a treatment plan to correct shortened adaptive tissue and is typically for the long-term restoration process. It must be done day after day after day. If they have a neurological diagnosis like Parkinson's Disease, Cerebral Palsy, CVA (stroke), Multiple Sclerosis, closed head injury, spinal cord injury, Traumatic Brain Syndrome, Huntington's Chorea, or end stage Alzheimer's Disease, they will typically present with the compounded problem of neurological tone. These devices are appropriate for both.

Correcting shortened adaptive tissue requires a device that will apply prolonged low load passive stretch. If a device is not designed to be able to be readjusted or remolded to continually work the patient's joint(s) toward correction, the device is not restorative. If a splint does not offer the appropriate amount of positioning stability, it is not rehabilitation. Again, these devices do both rehabilitation and restorative.

“Off the shelf” or prefabricated devices that truly have remoldable properties are adequate for most shortened adaptive tissue lost range of motion corrective treatments.

Tone requires a desired amount of flexibility to not make tone worse. High degrees of tone require the fingers to be able to repeatedly flex to a greater degree while the device maintains its memory to repeatedly take the patient back to the degree where the device was set. A moldable Kydex® material is used to form the splint to the desired position. Kydex® is a registered trademark of the Kleerdex Company LLC and is an extremely durable thermoplastic (thermal setting) alloy generally extruded in sheet form. The thickness of the Kydex® of the base of the device is required to retain that memory so slits proved to be the best option according to our continual trials with these types of patients. Slits have also proven to be extremely effective in working through tone at the wrist area. It proved, in our experience, to make the Kydex® base last longer than a solid one or one with a more narrow width from side to side to try to achieve the same results.

If the patient presents with tone, that tone must be “worked through” to reach muscle inhibition, or at least be dealt with appropriately, in order to reach any shortened adaptive tissue. The longer the patient goes without appropriate treatment, the higher the degree of lost range of motion and all the conditions that result from it.

It should be common knowledge for any clinician who works in this field that to use rigid or static splints on a patient who suffers from neurological tone will likely result in a continual “kicking in” of episodes of tone. This will cause the patient to loose more range of motion and may actually harm them. Alert and oriented patients have described an episode of tone as a horrible “Charlie Horse” or “muscle cramp” that continues for hours.

It is still common practice in the United States for restorative patients to receive splints that do nothing but act as a palm guard or attempt to prevent a patient's condition from worsening. Again, if the splints are not able to, in some fashion, be adjusted to increase and hold range of motion, they will not restore range of motion. If they are rigid or static, they may actually cause harm to a patient with neurological tone—and certainly may cause their range of motion to worsen.

Much effort has been expended to design hand splints that can be made available on a large scale in the prefabricated category in different sizes that can be heat molded during the actual fitting process to accommodate most patients. These are then designed to be heated and remolded at appropriate intervals to continue to correct a patient's lost range of motion due to adaptive tissue shortening and/or neurological tone. The most severe thumb adduction with Thenar Eminence Collapse may still require a custom cut out to allow for that condition if heat molding alone would still allow a potential for tissue trauma to the patient.

The orthosis or splint of the present invention can contain multiple features. All contain a moldable thermoplastic insert or core surrounded by covering of padding material. The insert has slits between the digits of the hand so that the insert can be molded for the individual digits. This padded splint goes underneath the hand, wrist, and forearm (ventral side). A separate moldable thermoplastic insert, again surrounded by padding, can form a dorsal portion to go opposite the splint on the back of the hand, wrist, and forearm. A plurality of straps attach to the padding of the underneath splint. Cylindrical shaped rolls can be attached to the insert of the splint and work with elastic straps on the splint to assist with correcting hyperextension of a joint or joints. In more detail, these important features of the splints are as follows:
a. Slits between fingers:
   i. allow for the fingers to move through tone and reach muscle inhibition instead of being rigid to work against their neurological tone. The Kydex allows them to pull inward then extends them back to the preset degree that was molded.
   ii. allows the base of each finger/thumb to be molded to fit that appendage to address each digit individually, typically decreasing the time required to work digits toward correct alignment.
b. slits in the center of the device running proximal to distal allow the wrist to move through periods of tone.
c. Rolls (Ventral Phalangeal Flexion Assists) placed just proximal to the hyperextended joint.
d. Rolls work with the straps (Dorsal Phalangeal Flexion Assists or DPFAs) to create flexion at the joint therefore correcting the hyperextension. The DPFAs are made of Elastic straps that fit over padded straps (that protect the skin) and close with a hook and loop (Velcro®) connector. These are sewn in distal to the hyperextended joint. They work in concert to create a 3-point leverage to encourage the joint into flexion.
e. On those hand orthoses made for people who present with Ulnar Drift, straps are sewn to the device to create 3-point leverage to correct that deformity. Typically straps are sewn to a hand splint going from medial to lateral. Ulnar Drift originates either from the wrist or from the MetaCarpal Phalanges joint(MCPs or “knuckles”). These straps are sewn in at the forearm going lateral to medial; at the MCP/back of hand and over end of the fingers going lateral to medial; at the wrist going medial to lateral with an extra pad sewn in at the medial aspect of the wrist to give extra protection and an extra push toward correction.

### BRIEF DESCRIPTION OF THE DRAWINGS

A better understanding of the present invention will be had upon reference to the following description in conjunction with the accompanying drawings, wherein:

FIG. 1 shows a side view of a first moldable thermoplastic splint insert;

FIG. 2 shows a top view of the insert of FIG. 1;

FIG. 3 shows a side view of a second moldable thermoplastic splint insert;

FIG. 4 shows a top view of the insert of FIG. 3;

FIG. 5 shows a pictorial view of a first splint of the present invention, the insert removed from the padding;

FIG. 6 shows a pictorial view of a second splint of the present invention, the insert removed from the padding;

FIG. 6a shows a pictorial view of the insert from FIG. 6 with alternate rolls placed;

FIG. 7 shows a pictorial view of the dorsal portion, the insert removed from the padding;

FIG. 8 shows a pictorial view of a third splint of the present invention having a dorsal portion, the inserts removed from the padding;

FIG. 9 shows a pictorial view of the splint of FIG. 5 in use with a dorsal portion;

FIG. 10 shows a pictorial view of a fourth splint of the present invention having a dorsal portion;

FIG. 11 shows a top pictorial view of a fifth splint of the present invention;

FIG. 12 shows a side pictorial view of the splint of FIG. 11 being worn;

FIG. 13 shows a bottom pictorial view of a sixth splint of the present invention showing the opening for the insertion of the moldable thermoplastic insert;

FIG. 14 shows a top pictorial view of the sixth splint of the present invention showing how the splint would be worn;

FIG. 15 shows the splint of FIGS. 13-14 with the dorsal portion 50 on the back of the hand, wrist, and forearm, and one of the straps attached;

FIG. 16 shows the splint of FIGS. 13-15 with all straps connected;

FIG. 17 shows a top pictorial view of a seventh splint of the present invention; and,

FIG. 18 shows a top pictorial view of an eighth splint of the present invention.

### DETAILED DESCRIPTION OF THE PREFERRED EMBODIMENT

With reference to FIGS. 1-10, various splints of the instant invention and the features of the splints are shown.

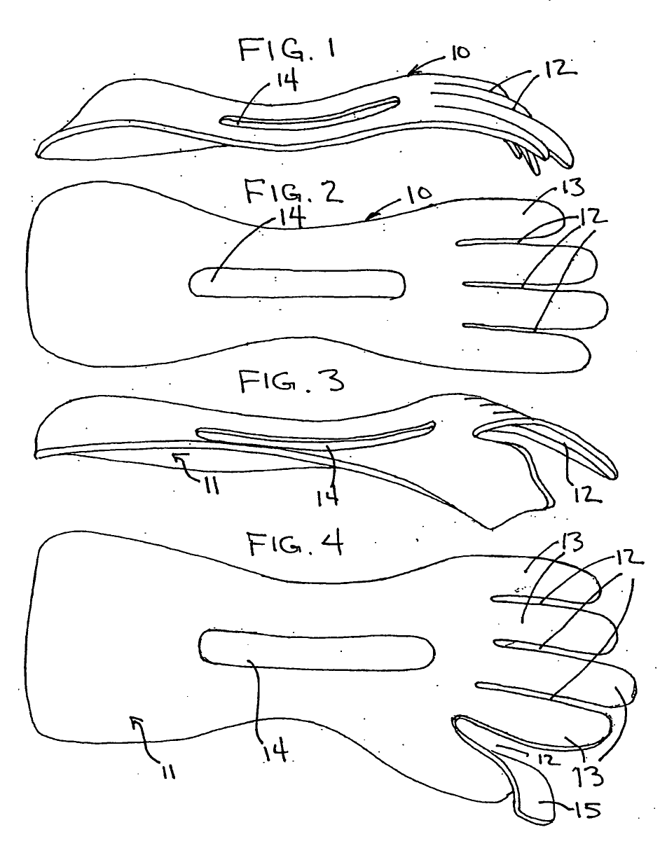

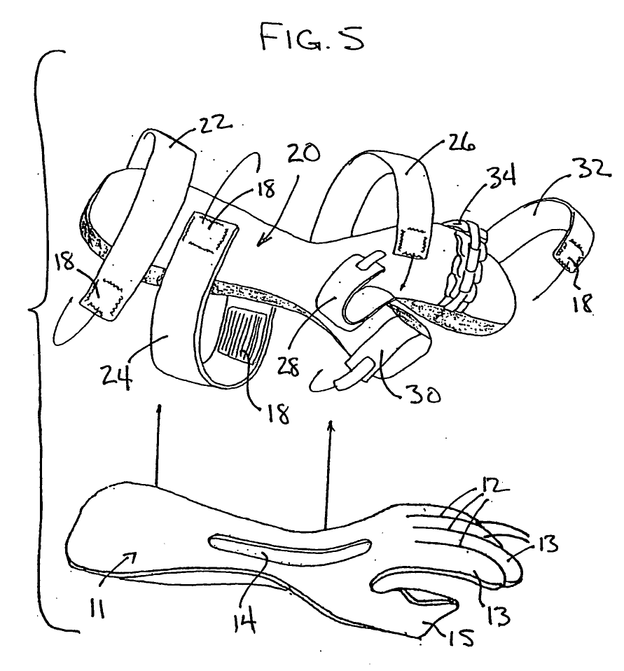

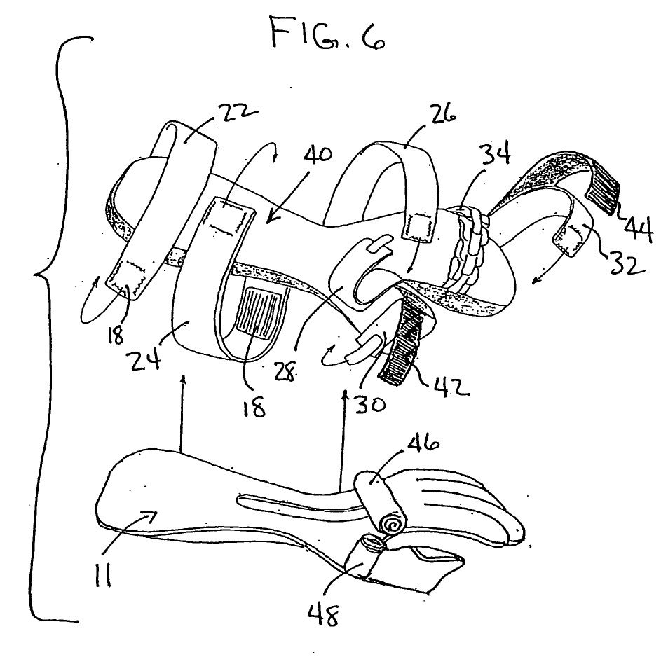

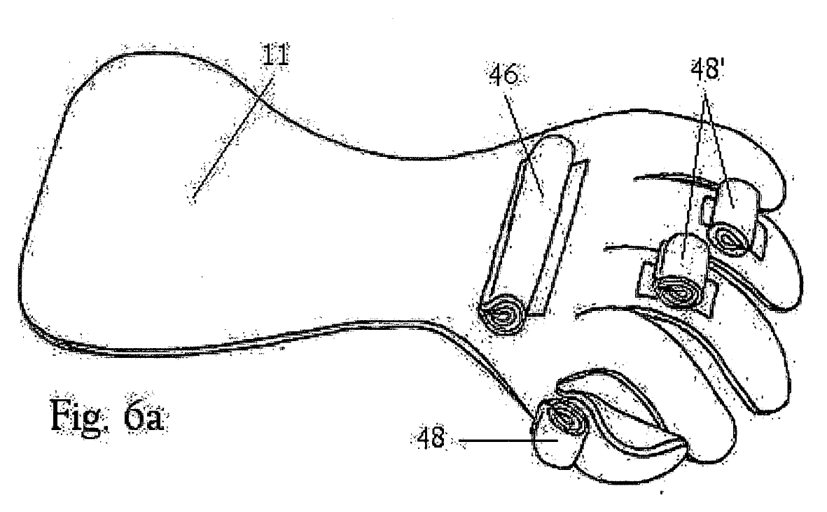

FIGS. 1 and 2 show side and top views of a first moldable thermoplastic insert 10 and FIGS. 3 and 4 show side and top views of a second moldable thermoplastic insert 11. These inserts 10 and 11 contain slits 12 between the fingers 13 and thumb 15 and also have a central opening 14, all of which make the insert more dynamically moldable to a desired shape. There are two thumb versions of this invention: The thumb 15 is an extension of the Kydex® plastic that is totally enclosed in padded material and may be molded to meet almost any degree of range of motion for correction. This version is desired when a secure base is required to address hyperextension of any joint of the thumb to be able to create the three-point leverage with the “rolls” just proximal to the hyperextended joints and the straps with elastic properties just distal to work the joint(s) toward flexion, therefore correcting the hyperextension. If the thumb is in severe flexion the clinician may prefer the version of the device with a fabric pocket (not shown) sewn in at the appropriate position for the flexed thumb. Into this pocket any type of padding material may be inserted to create the desired safe abduction. Gradually increasing the bulk of the padding gradually increases the abduction toward normal alignment. Enclosed with this style is a piece of dense moisture resistant foam that can be trimmed to meet many degrees of thumb adduction. A strap may be sewn into this pocket to wrap around the thumb to hold it in place.

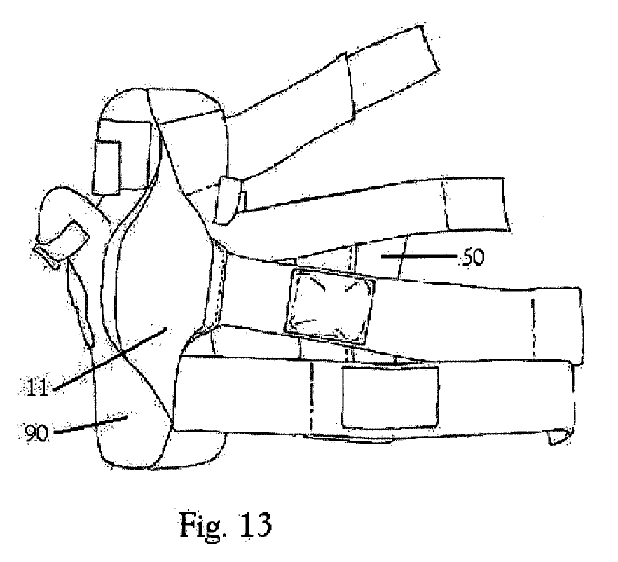

FIG. 5 shows an exploded pictorial view of a splint 20. The thermoplastic insert 11 has been removed from the padded splint 20 for ease of understanding, but, in use, the insert 11 is contained within the padded covering of the splint 20. The padded covering of the splint 20 can contain a lengthwise opening for insertion of the insert 11 into the splint 20, which opening can be closed by the use of a hook and loop fastening system (see FIG. 13). This opening would be on the opposite side of the splint 20 from that which will be placed against the wearer. These hand splints, such as splint 20, are prefabricated and will all be shipped to the patient in a neutral position, as shown.

Splint 20 is shown with a forearm strap 22 and a proximal MCP strap 26, which go lateral to medial, and a wrist strap 24, which goes medial to lateral. This strap 22, 24, 26 configuration is designed to aid in the correction of ulnar drift. The splint 20 also includes MP thumb strap 28, distal thumb strap 30, and distal MCP strap 32. Finger separators 34 are also included. Each of the straps 22, 24, 26, 28, 30, 32 is fixedly attached to the splint 20 on one end and the other end of each strap can be removably attached to the fabric of the splint 20 using a hook and loop fastening system at a desired location for the particular wearer. Also, the finger separators 34 are removably attachable to the splint 20 using a hook and loop fastening system so that they can be placed as needed for the particular wearer.

The splint 40 of FIG. 6 is similar to the splint 20 of FIG. 5, but includes four important additions. Straps 22, 24, 26, 28, 30, 32, and finger separators 34 are the same as in splint 20. The insert 11 of splint 40 is shown having finger roll 46 and thumb roll 48 attached thereto using a hook and loop fastening system. In combination with an elastic distal MCP strap 44 and an elastic distal thumb strap 42, these rolls 46, 48 assist in correcting hyperextension of the MCP joints and are ventral phalangeal flexion assists. Elastic straps 42, 44 are also fixedly attached at one end and removably attached at the other end using a hook and loop fastening system. FIG. 6a shows placement of additional distal finger rolls 48′.

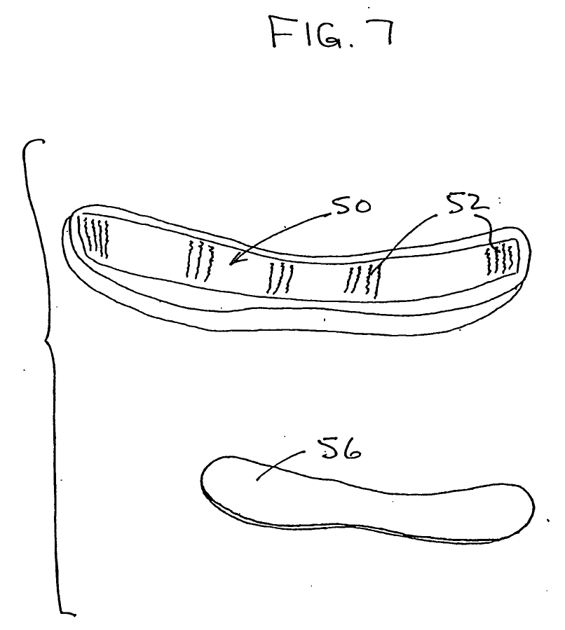

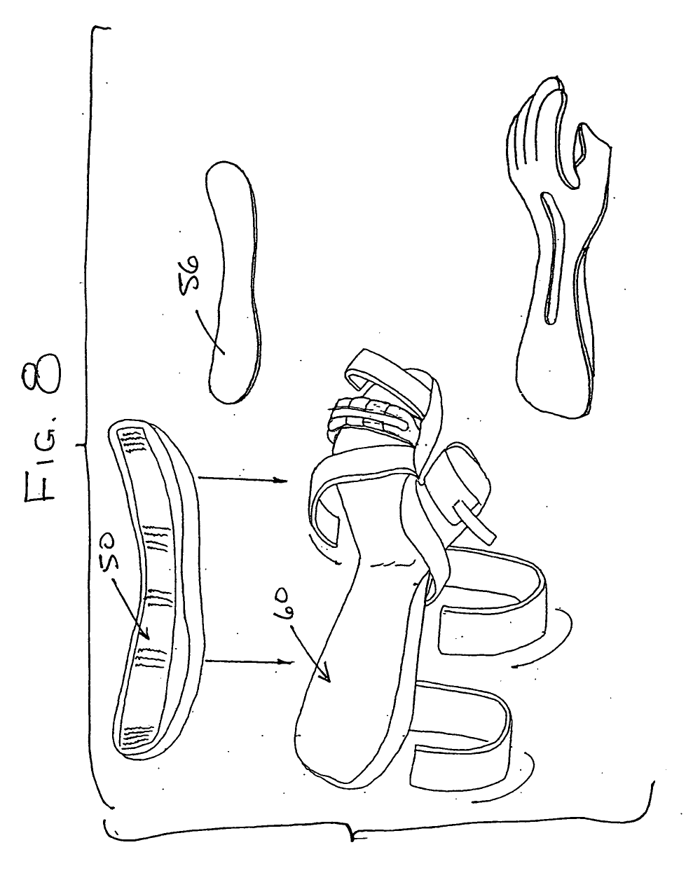

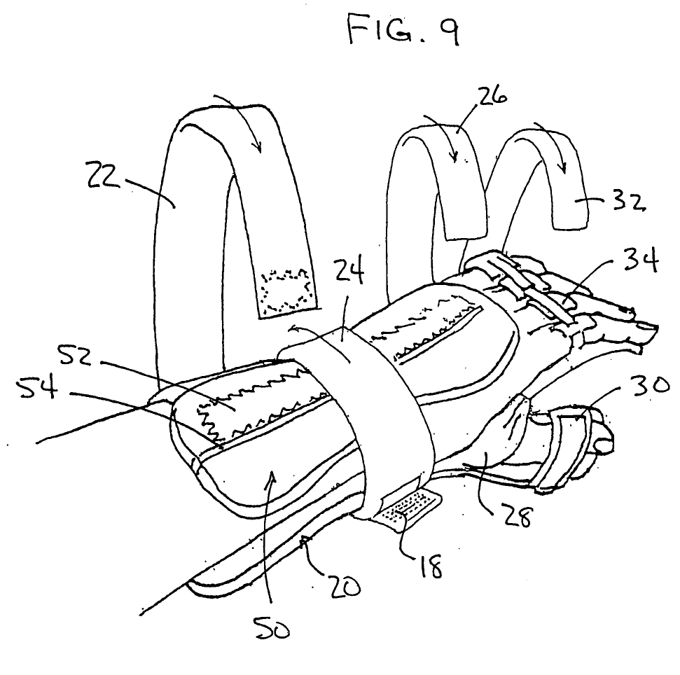

FIG. 7 shows an exploded pictorial view of the dorsal portion 50 used with the various splints, the moldable thermoplastic insert 56 being shown removed from the portion 50. As will be described in the description of other Figures, this dorsal portion 50 is placed opposite the splint on the back of the hand, wrist, and forearm of the wearer. Portion 50 includes hook and loop fastening material 52 so that the splint straps, for example straps 22, 24, 26 of splints 20 and 40, will maintain portion 50 in its desired position.

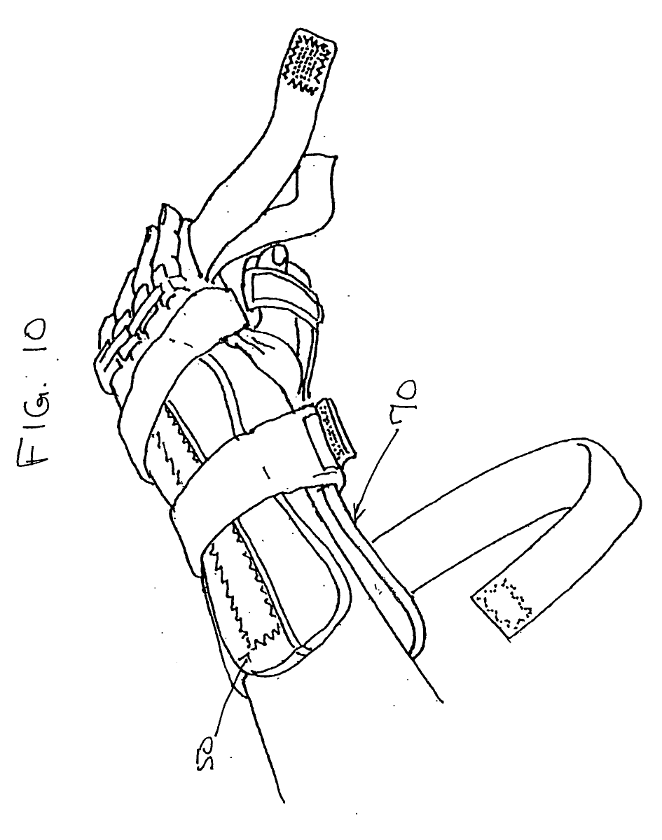

FIG. 8 shows an exploded pictorial view of a third splint system 60.

FIG. 9 shows a pictorial view of the splint 20 of FIG. 5 in use, the splint 20 also including a dorsal portion 50.

FIG. 10 shows a pictorial view of a fourth splint system 70.

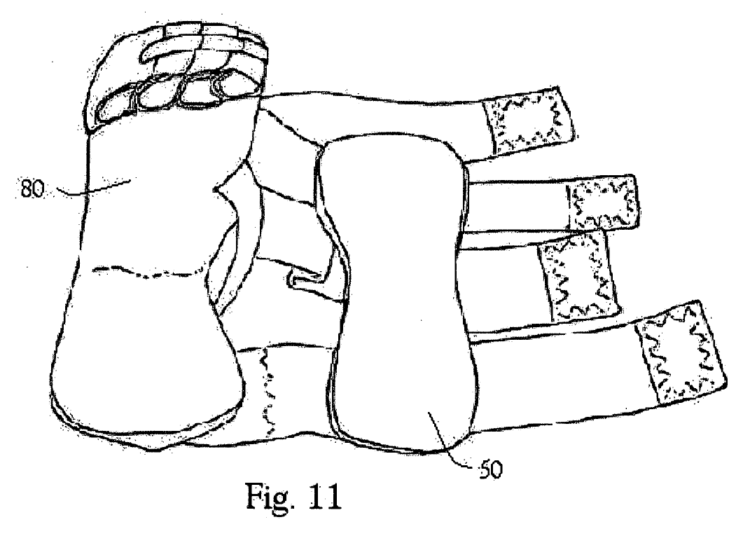

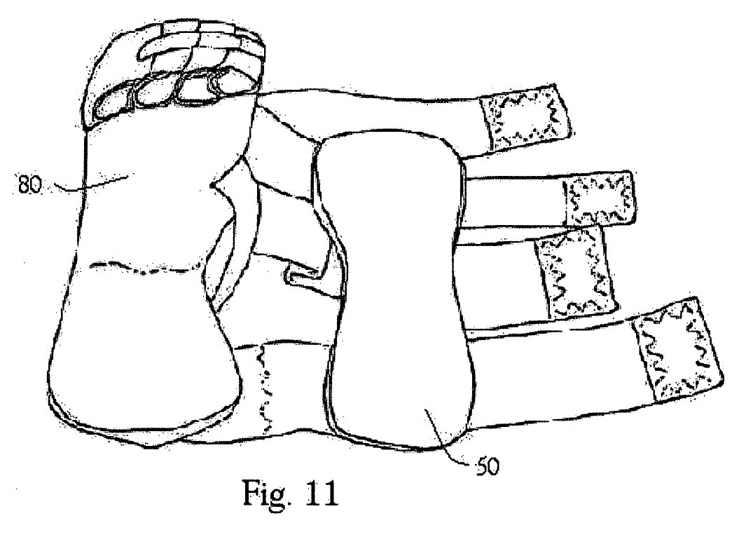

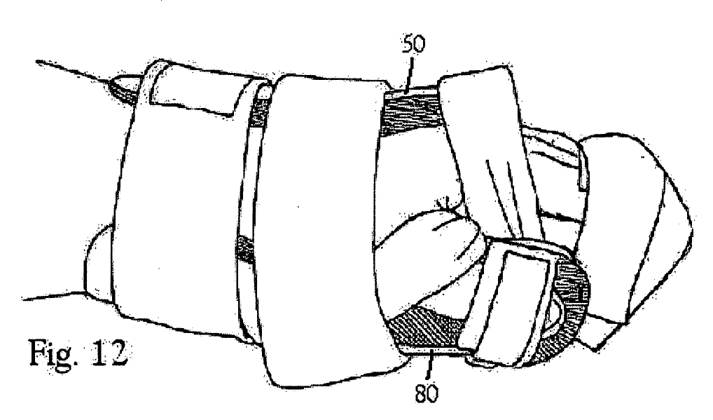

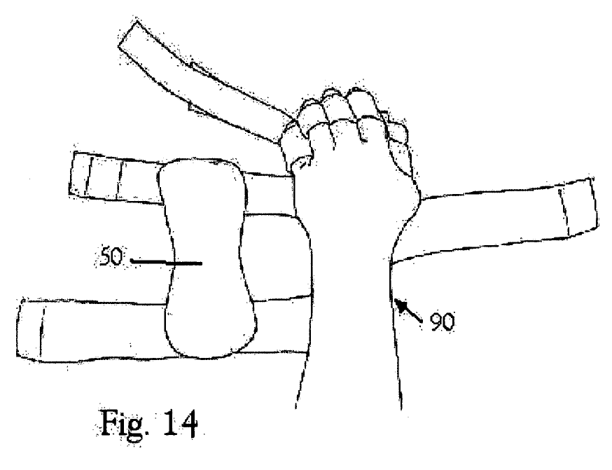

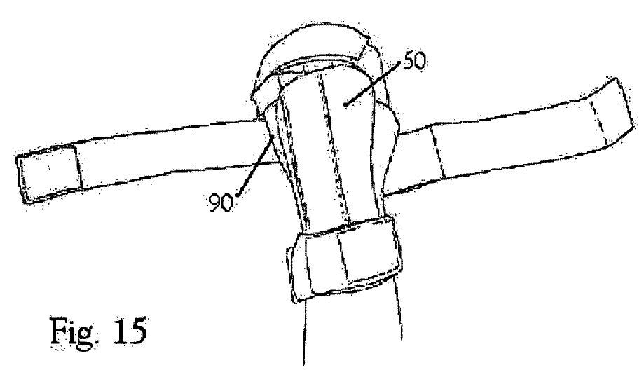

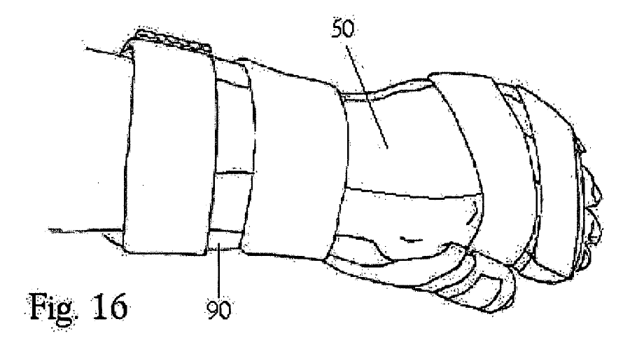

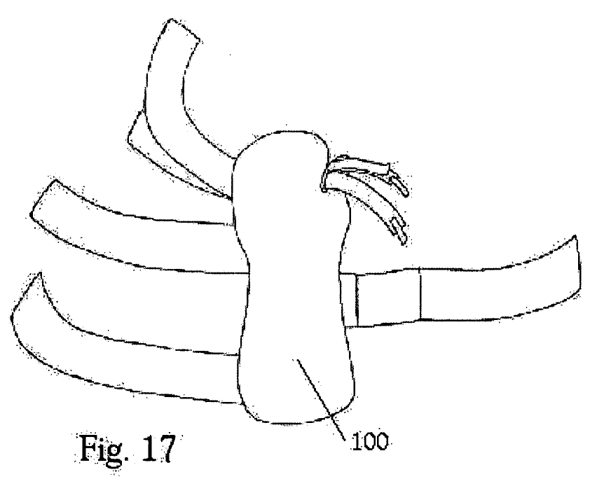

With reference to FIGS. 11-18, the following examples explain four possible splint configurations 80 (FIGS. 11-12), 90 (FIGS. 13-16), 100 (FIG. 17), and 110 (FIG. 18).

The splint 80 of FIGS. 11-12 is branded by applicant as the HyperHand™ Dorsal with INFINITY. The INFINITY, or the dorsal portion 50, will allow the patient's wrist to “work through” episodes of tone while always easing it back to the molded degree of range of motion.
a. Hyper extended wrist—INFINITY to treat. Mold to sandwich the patient's hand between it and the opponen of the HyperHand™. Remold both the base and the INFINITY to “sandwich” the patient's hand between them as the patient's range of motion improves to continually move toward normal alignment.
b. Slits between each digit will allow the fingers to also work through their tone.
c. Ulnar Drift-Type of Finger Separators are designed to attach, such as by Velcro®, to each side to allow adjusting as the patient's condition improves and can be turned to anchor them medial to lateral or lateral to medial. They are also long enough to treat fingers that cross over or lap over each other. The material was chosen for its value in working with extremely delicate skin issues. Each individual portion of the attachment that separates each finger has a hook and loop tab sewn to the end of it. This allows the fingers to be securely positioned to correct alignment as each one is anchored to the next. Because the entire attachment is designed with hook and loop to attach it to the padding of the device, it can be adapted to correct Ulnar Drift, overlapping/malaligned fingers and to separate and pad those that press against each other.

The splint 90 of FIGS. 13-16 is branded by applicant as the HyperHand™ Dorsal with INFINITY for Ulnar Drift
a. Hyper extended wrist—INFINITY to treat.
b. Ulnar Drift—straps provide the required 3-point leverage to correct.
   i. Middle point is at wrist where there is an extra pad to protect the bony area while providing an extra “push” toward correction going medial to lateral.
   ii. Distal strap at fingers is placed to gradually encourage the fingers from lateral to medial.
   iii. Forearm strap is the proximal focal point of the 3-point leverage and is sewn in to go lateral to medial.
   iv. Ulnar Drift-Type of Finger Separators are designed to attach, such as by Velcro®, to each side to allow adjusting as the patient's condition improves and can be turned to anchor them medial to lateral or lateral to medial They are also long enough to treat fingers that cross over or lap over each other. The material that has foam laminated to one side was chosen for its value in working with extremely delicate skin issues. The completed separators must be thin enough to work between tight fingers, padded and of a smooth material to protect the skin and be comfortable, and wide enough to address hyperextension of various joints of the thumb and fingers.
c. Hyperextension of various joints of the thumb and fingers.
   i. Ventral Interphalangeal Flexion Assist ROLLS are placed on the Kydex® base of the opponen under the padding. These are placed just proximal to the hyper extended joints.
   ii. Dorsal Interphalangeal Flexion Assist Straps have elastic properties and are placed distal to the hyper extended joints to work with the ROLLS to bring the joint(s) into flexion—thereby correcting the hyperextension. These two attachments work in concert to create the three-point leverage required of restorative orthotics. The elastic properties allow the patient's joints to “work through” the tone as they hyperextend and the straps bring them into flexion - repeatedly until the joint typically reaches muscle inhibition. This must occur in order to treat the underlying adaptive tissue shortening.
   iii. The present thinking is that fifteen to twenty minutes of continual flexing and extending, or the continual extending and being brought back to into flexion, uses the Gamma Motor System via the Golgi Tendon Organs to bring a condition of muscle inhibition to the affected area.
   iv. Once the tone response is weakened, the shortened tissue can be reached and prolonged low load passive stretch can be applied, with the same device, to begin the elongation or re-lengthening process.
   v. Slits in the opponen between each digit allow the appropriate movement of these digits to create the optimal therapeutic conditions. To block these movements with a rigid base and rigid dorsal components would exacerbate the tone and result in worsening the condition and pain to the patient.

The splint 100 of FIG. 17 is branded by applicant as the HyperHand™ for hyper extended joints of fingers and thumb.
a. Hyperextension of various joints of the thumb and fingers.
   i. Ventral Interphalangeal Flexion Assist “Rolls” are placed on the Kydex® base of the opponen under the padding. These are placed just proximal to the hyper extended joints.
   ii. Dorsal Interphalangeal Flexion Assist Straps have elastic properties and are placed distal to the hyper extended joints to work with the “Rolls” to bring the joint(s) into flexion—thereby correcting the hyperextension. The elastic properties allow the patient's joints to “work through” the tone as they hyperextend and the straps bring them into flexion—repeatedly until the joint typically reaches muscle inhibition. This must occur in order to treat the underlying adaptive tissue shortening.
   iii. Slits in the opponen between each digit allow the appropriate movement of these digits to create the optimal therapeutic conditions. To block these movements with a rigid base and rigid dorsal components would exacerbate the tone and result in worsening the condition and pain to the patient.

The splint 110 of FIG. 18 is branded by applicant as the HyperHand™ for Ulnar Drift
a. Ulnar Drift—straps provide the required 3-point leverage to correct.
   i. Middle point is at wrist where there is an extra pad to protect the bony area while providing an extra “push” toward correction going medial to lateral.
   ii. Distal strap at fingers is placed to gradually encourage the fingers from lateral to medial.
   iii. Forearm strap is the proximal focal point of the 3-point leverage and is sewn in to go lateral to medial.
   iv. Ulnar Drift-Type of Finger Separators are designed to attach, such as by Velcro®, to each side to allow adjusting as the patient's condition improves and can be turned to anchor them medial to lateral or lateral to medial. They are also long enough to treat fingers that cross over or lap over each other. The material was chosen for its value in working with extremely delicate skin issues.
   v. Slits in the opponen between each digit allow the appropriate movement of these digits to create the optimal therapeutic conditions. To block these movements with a rigid base and rigid dorsal components would exacerbate the tone and result in worsening the condition and pain to the patient.

The foregoing detailed description is given primarily for clearness of understanding and no unnecessary limitations are to be understood therefrom for modifications can be made by those skilled in the art upon reading this disclosure and may be made without departing from the spirit of the invention.

What I claim is:

1. A hand orthosis, comprising:
a ventral side splint positionable against at least a portion of a patient's forearm and extending distally therefrom substantially to the ends of each finger, the splint comprising a core portion and a covering, the core portion being made of a thermal setting remoldable material and being divided into individual finger portions by longitudinal slits, each finger being separately remoldable to a selected position and the material having a preselected inherent amount of flexibility and sufficient positional memory to return to the selected position after flexing; and
means for detachably attaching the splint securely to the patient's forearm and hand.

2. The hand orthosis of claim 1, wherein the covering includes padding on at least a side where the splint contacts the patient.

3. The hand orthosis of claim 1, wherein the attachment means includes at least one strap positioned adjacent to at least one of the patient's forearm, wrist, or fingers, the strap being secured at one end to the splint and extendable around the specific portion of the patient's arm and releasably securable at an opposite end.

4. The hand orthosis of claim 1, further comprising a dorsal side splint positionable against at least a portion of a patient's forearm and extending distally therefrom substantially to the metacarpal region of the patient's hand, the dorsal splint comprising a core portion and a covering, the core portion being made of a thermal setting material remoldable to a selected position and the material having a preselected inherent amount of flexibility and sufficient positional memory to return to the selected position after flexing.

5. The hand orthosis of claim 1, wherein the core portion of the ventral side splint includes a slot elongated in the proximal to distal direction in a region substantially adjacent to the patient's wrist and metacarpals to enhance the flexibility and remoldablity of the core portion in that region.

6. The hand orthosis of claim 1, further comprising an insert to produce a raised area on the splint against the patient positioned proximal to a hyperextended joint of a digit.

7. The hand orthosis of claim 6, further comprising at least one releasably securable strap for extending over at least one affected digit and positioned distal to the hyperextended joint, the strap being attached at one end to the splint and the opposite end being releasably secured to the splint.

8. The hand orthosis of claim 7, wherein the at least one strap is elastic to allow resisted movement of the digit in a direction way from the splint.

9. The hand orthosis of claim 1, further comprising straps secured at each end to separately secure each digit in place with respect to the splint for treatment of ulnar drift.

10. The hand orthosis of claim 9, wherein the straps are comprised of elastic to allow resisted movement of each digit separately in a direction way from the splint.

11. The hand orthosis of claim 9, wherein the attachment means includes straps positioned adjacent to the patient's forearm, wrist, and fingers distal to the metacarpal-phalanges joint, the straps being secured at one end to the splint, extendable around the specific portion of the patient, and releasably securable at an opposite end to the splint, thereby providing a three-point leverage to treat ulnar drift.
